(19) United States
(12) Patent Application Publication
(10) Pub. No.: US 2021/0128836 A1
(43) Pub. Date: May 6, 2021

(54) POWER UNIT FOR USE IN AN AUTOINJECTOR AND METHOD OF ASSEMBLING SUCH POWER UNIT

(71) Applicant: Novo Nordisk A/S, Bagsvaerd (DK)

(72) Inventor: Ebbe Kiilerich, Copenhagen NV (DK)

(21) Appl. No.: 16/972,299

(22) PCT Filed: Jun. 5, 2019
(86) PCT No.: PCT/EP2019/064737

§ 371 Date: Dec. 4, 2020

(30) Foreign Application Priority Data
Jun. 5, 2018 (EP) 18176176.8

(51) Int. Cl. A61M 5/20; A61M 5/315

(52) U.S. Cl. CPC A61M 5/2033; A61M 2205/43; A61M 2207/00; A61M 5/3157

(57) ABSTRACT

A power unit (15) for use in an autoinjector (10) for driving a plunger (500) distally along an axis in an expelling movement so as to expel a drug from a held drug container, the power unit (15) comprising: a) a plunger (500) comprising retaining geometries (515) and defining radially protruding click protrusions (525), b) a drive spring (550) arranged in a tensed state with a force biasing the plunger (500) distally, and c) a power base (400) coupled to the drive spring (550) and the plunger (500), the power base (400) comprising a base part grounding the drive spring (550), and retaining elements (410) releasably engaging retaining geometries (515) of the plunger (500), the power base (400) further defining a resilient click arm (410) configured to cooperate with the click protrusions (525) of the plunger (500), the click arm (410) being moved radially to provide a click noise for each click protrusion that passes the click arm during the expelling movement, wherein the click arm (410) comprises a straining surface geometry (415d) arranged to cooperate, in a preparing step, with a tool (20) arranged distally relative to the click arm for moving the click arm (410) radially into a tensed state enabling the plunger (500) to be moved proximally. A method of assembling the power unit (15) is further disclosed.

### FIELD OF THE INVENTION

The present invention relates to injection devices for injecting a medicament. In particular the present invention relates to a power unit for use in an autoinjection device having a releasable plunger, a click arm, and a radially protruding click protrusion on the plunger to generate a click during expelling. The present invention further relates to a method of obtaining a simplified process for assembling such device.

### BACKGROUND OF THE INVENTION

In relation to some diseases patients must inject a medicament on a regular basis such as once weekly, once daily or even a plurality of times each day. In order to help patients administering one or more doses of a medicament injection devices, such as autoinjectors, are widely used. Some injection devices generate feedback signals to signify certain operational states during operation of the device. For example, injection devices may generate one or more click sounds during the expelling procedure, signalling initialisation, finalization or progression of the expelling. References WO 2012/022810, WO 2016/089871 and WO 2017/191159 all provide disclosure of such devices wherein sound activating geometries associated with a plunger cooperate with additional elements for generating click sounds as the plunger is driven forward.

Typically, during manufacturing of injection devices, smaller or larger sub-assemblies of components are initially formed which are subsequently interconnected or coupled with additional components. Autoinjectors, such as disclosed in U.S. Pat. No. 6,099,503 and WO 2017/191159, may include a power unit comprising a drive spring which is brought in an energized state providing a force for driving forward a plunger, wherein the drive spring is arranged in a pre-strained state during an early assembly step. As click sounds typically are generated by having a click element rapidly snapping during forward movement of the plunger relative to the click element this condition typically makes the movement possible only in the expelling direction. Hence, inclusion of click sound generating elements in such devices typically lead to an increase in complexity both having regard to the number of components, and to the number of assembly steps when manufacturing the autoinjector.

Having regard to the above-identified prior art devices, it is an object of the present invention to provide a power unit for use in an autoinjector which is less complex and wherein the assembly procedure for forming the power unit, and ultimately the autonijector, is simplified.

### BRIEF DESCRIPTION OF THE INVENTION

In a first aspect the present invention relates to a power unit for use in an autoinjector, the power unit being configured for driving a plunger distally along an axis in an expelling movement so as to expel a drug from a held drug container, the power unit comprising:
a plunger having an elongated shape extending along said axis, the plunger comprising one or more retaining geometries and defining one or more radially protruding click protrusions,
a drive spring arrangeable, in a preparing step, in a tensed state wherein a first end of the drive spring acts on the plunger with a force urging the plunger distally, and
a power base operably coupled to the drive spring and the plunger, the power base comprising a base part grounding the drive spring at a second end, and one or more retaining elements each releasably engaging a respective one of the one or more retaining geometries of the plunger to retain the plunger against the force of the drive spring, the power base further comprising a resilient click arm configured to cooperate with said one or more click protrusions of the plunger, the click arm being moved radially to provide a click noise for each click protrusion that passes the click arm during the expelling movement, wherein cooperating surfaces of the click arm and the respective click protrusions define, in the direction of the expelling movement, a leading surface pair that gradually increases tension in the click arm followed by a trailing surface pair that abruptly releases tension in the click arm to provide said click noise, and wherein the click arm further comprises a straining surface geometry arranged to cooperate, in the preparing step, with a tool arranged distally relative to the click arm for moving the click arm radially into a tensed state enabling the plunger to be moved proximally while allowing the surfaces of said trailing surface pair to pass each other during tensioning of the drive spring.

By configuring the straining surface geometry of the click arm to be engaged with a tool during an assembly step, the click arm may be strained by being brought sufficiently out of reach relative to cooperating elements of the plunger to enable the plunger to be moved proximally relative to the power base during tensioning of the drive spring. In prior art devices, click elements, such as click arms that cooperate with protrusion elements to create click sounds typically dictates one-way movement between the click arm and the cooperating protrusions which implies a particular way of orienting and moving the implied components during assembly. For example, in prior art devices wherein additional components are used, the plunger will be initially arranged relative to the click arm so that the plunger approaches the click arm from the proximal side of the click arm, i.e. the plunger is moved distally relative to the click arm. This implies a pronounced simplification of the assembling procedure and enables the autoinjector to be more cost effectively provided.

In certain embodiments, each of the surfaces of the trailing surface pair comprises an abrupt sloping surface, whereas at least one of the surfaces of the leading surface pair comprises a gradually sloping surface. In some embodiments, one of the surfaces of the leading surface pair comprises abrupt sloping surface whereas in other embodiments, both the surfaces of the leading surface pair comprise a gradually sloping surface. In certain embodiments, each of the surfaces of the trailing surface pair comprises an abrupt sloping surface. Exemplary embodiments may include surfaces of the trailing surface pair being arranged substantially orthogonal to the axis, such as being arranged with in inclination angle larger than 70 degrees, preferably larger than 80 degrees, and more preferably larger than 85 degrees relative to the axis. The abruptly sloping surfaces of the trailing surface pair ensures that a particularly powerful click sound is generated each time the click arm passes a radially protruding click protrusion of the plunger.

In some embodiments, the click arm assumes a first relaxed radially inwards located position when not being acted upon by other components. In such embodiments, the click arm is movable into a tensed second radially outwards located position, such as when being acted upon by the one or more radially protruding click protrusions of the plunger, and when acted upon by the tool.

In some embodiments, the straining surface geometry of the click arm includes a distally oriented inclined surface having a surface normal inclined relative to the axis so that the surface normal intersects with the axis, and wherein said tool includes a reaction surface so oriented that, in the preparing step, the reaction surface engages the distally oriented inclined surface of the click arm thereby forcing the click arm to move radially outwards as the tool is moved proximally relative to the power base. A particularly simple construction is thereby obtained.

In other embodiments the straining surface geometry of the click arm includes a surface geometry, such as a stepped surface arranged at distal facing surface, wherein the stepped surface is engageable by a tool, and wherein the tool forces the click arm radially outwards as it cooperates with a non-circular outwards facing tool surface, in the course of the tool being rotated around the axis from a first rotational position wherein the click arm is in a radially inwards relaxed state and into a second rotational position wherein the click arm is in a radially tensed state.

In some embodiments the power unit further comprises a trigger element being movable relative to the power base between a first position and a second position, wherein, when the trigger element assumes the first position, the trigger element engages with the one or more retaining elements to maintain retaining engagement between respective ones of the one or more retaining elements and the one or more retaining geometries of the plunger. When the trigger element assumes the second position, the trigger element allows the one or more retaining elements to release said retaining engagement allowing the plunger to move distally.

In some embodiments the plunger comprises a spring seat, and wherein the drive spring is a compression spring having the second end of the drive spring grounded by the power base and the first end providing a distally directed force on the spring seat of the plunger.

In further embodiments the power base further comprises a distally facing spring seat arranged to receive the second end of the drive spring. In some embodiments, the spring seat and the click arm of the power base are formed as a unitary component, such as being molded in a molding process.

In still further embodiments, the power base is formed to define a closed proximal end cap configured for being coupled to the proximal end of a sleeve shaped housing of the autoinjector. The power base may in certain embodiments include a sleeve shaped power base housing that is formed to encircle at least a proximal end portion of the plunger and at least a proximal end portion of the drive spring, where the sleeve shaped power base housing is formed to protrude axially in the distal direction from the closed proximal end cap and extending further distally than the one or more retaining elements. In accordance with the first aspect, the power base housing is shaped with a passage so as to allow the said tool to be axially inserted radially within the power base housing but radially outside the plunger and the drive spring to enable engagement with the straining surface geometry of the click arm. In some embodiments, the power base housing includes guiding surfaces adapted for guiding the trigger element as it moves relative to the power base between the first position and the second position.

In further embodiments the click arm is formed in one piece with a transverse section forming a distally facing spring seat arranged to receive the second end of the drive spring. By forming the click arm and the spring seat as a unitary component, the power unit and the assembling procedure for preparing the power unit may be simplified and enables a more cost-effective and robust power unit to be provided.

In further embodiments, the power base of the power unit comprises a transverse section and a spring guide that is arranged to extend axially in distal direction relative to the transverse section, the spring guide and/or the transverse section defining a distally facing spring seat that receives the second end of the drive spring, and wherein the click arm is formed in one piece with the transverse section and/or the spring guide.

In other embodiments, the power base defines a distal facing spring seat that receives the second end of the drive spring, and wherein the power base defines a single unitary component having the spring seat formed in on piece with the click arm, and optionally, formed in one piece with the one or more retaining elements. In some embodiments, the spring seat of the power base is arranged distally to the one or more retaining elements. In other embodiments, the spring seat of the power base is arranged proximally relative to the one or more retaining elements.

In still further embodiments the power base comprises a transverse section and a spring guide that extends distally from the transverse section, the spring guide and the transverse section defining a distally facing spring seat arranged to receive the second end of the drive spring.

In some embodiments the spring guide is formed in one piece with the transverse section to form a distally facing cavity coaxially receiving the second end of the drive spring. In other embodiments, the spring guide is formed in one piece with the transverse section, and wherein the spring guide defines a rod-shaped element configured to be received coaxially within the drive spring.

In further embodiments, the said click arm provides a first click arm and wherein the power base comprises one or more additional click arms configured similarly to the first click arm to cooperate with click protrusions of the plunger, and wherein the plurality of click arms are arranged symmetrically around the plunger. The number of click arms may in different embodiments be one, two, three, four or more individual click arms. The click arms may in certain embodiments be provided as axially extending arms which generally runs parallel with the axis. In certain embodiments, each of the click arms extends distally from a proximally arranged transverse section. In other embodiments, each of the click arms extends proximally from a distally arranged tubular section of the power base.

In some embodiments respective ones of the one or more retaining elements, such as being provided as retaining arms, is defined by one of said click arms. In other embodiments the one or more click arms are formed individually from the one or more retaining elements.

In some embodiments the plunger is hollow, wherein the drive spring is a compression spring, and wherein the compression spring extends at least partly into the hollow plunger. In other embodiments, the drive spring is provided as a helical compression spring arranged to encircle at least a portion of the plunger. When the drive spring of the power unit is arranged in the tensed state, the compression spring is arranged in a compressed state and thus urges the ends of the drive spring away from each other.

In further embodiment, the power unit is coupled with a housing, and with a drug container to provide an autoinjector. In still further embodiments, the power base defines a proximal end cap to be received within a sleeve formed housing at the proximal end thereof.

In a second aspect, the invention relates to a method of preparing, such as assembling, a power unit as defined in any of the embodiments described in connection with the first aspect mentioned above. The method comprises the steps of:
a) providing the plunger, the power base, and the drive spring, wherein the drive spring is provided in the form of a helical compression spring,
b) inserting the plunger into a tool arrangement whereby the plunger is arranged along the axis and is supported at its distal end by a first tool part,
c) arranging the drive spring concentrically with the plunger in axially overlapping relationship with the plunger,
d) arranging the power base concentrically with the drive spring with the straining surface geometry of the click arm being engaged by a second tool part,
e) moving the power base and the second tool part relative to each other to thereby move the click arm radially into a tensed state thereby enabling the plunger to be moved proximally while allowing the surfaces of said trailing surface pair to pass each other,
f) tensioning the drive spring between the power base and the plunger by moving the plunger in the proximal direction allowing the surfaces of said trailing surface pair to pass each other, and
g) arranging the one or more retaining elements in releasable engagement with the respective retaining geometries of the plunger to retain the plunger relative to the power base against the force of the drive spring.

In some embodiments, the method of preparing a power unit is characterized in that, in step d), the second tool part includes a reaction surface configured to engage said straining surface geometry of the click arm with the distally oriented inclined surface, and wherein, in step e), due to the engagement between the distally oriented inclined surface with the reaction surface, the click arm moves radially into a tensed state to enable the plunger to be moved proximally while allowing the surfaces of said trailing surface pair to pass each other.

The second tool part may in some embodiments the first and second tool parts are arranged distally relative to the click arm of the power base. The tools may be so formed that the second tool part includes an opening through which the plunger may protrude.

In some further embodiments, the method further comprises the steps of:
h) providing the trigger element, and arranging the trigger element relative to the power base, and
i) subsequent to step g), moving the trigger element into the first position so that the trigger element engages with the one or more retaining elements to maintain retaining engagement between respective ones of the one or more retaining elements and the one or more retaining geometries of the plunger.

In further embodiments, the power unit according to the first aspect and the power unit prepared by the method according to the second aspect may include any of the features and combination of features disclosed in the following.

In some embodiments, the autoinjector is configured for expelling a dose of drug from a held drug container, the injection device comprising:
a housing having a proximal end (P) and a distal end (D),
a drug container comprising a container barrel and a piston that is sealingly and slideably arranged inside the container barrel,
an injection needle connected to or connectable to a distal end of the drug container,
a plunger adapted for cooperation with the piston to drive the piston distally along a central axis, the plunger comprising a retaining geometry,
an energy source, such as the drive spring, coupled to the plunger and providing a force on the plunger in a distal direction,
a plunger retaining arrangement comprising a retaining element that engages with the retaining geometry to retain the plunger in a pre-firing position, the retaining element being movable in a radial direction to release said engagement,
a user operable trigger element cooperating with the retaining element and shiftable from a pre-firing condition wherein the trigger element cooperates with the retaining element to maintain retaining engagement with the retaining geometry of the plunger, and into a firing condition wherein release of the retaining engagement is initialized,
   wherein the autoinjection device defines a memory element being movable from a pre-firing position to a fired position, the memory element comprising an engagement surface configured for sliding engagement with an activation surface of the retaining element, wherein at least one of the engagement surface and the activation surface includes a surface being inclined relative to said radial direction, and
   wherein, upon the trigger element being shifted from the pre-firing condition to the firing condition, the energy source acts on the plunger to force the retaining element radially to release the retaining engagement, the radial movement of the retaining element in turn forcing the memory element to move into the fired position by sliding engagement between the activation surface of the retaining element and the engagement surface of the memory element, said movement of the memory element being induced by said surface being inclined relative to said radial direction.

As the energy source acts to force the memory element to move into the fired position the autoinjection device becomes less dependent on tolerance variations so that the correct functions of a secondary function controlled by the position of the memory element becomes more reliable. In addition, the movement of the memory element into the fired position means that the movement of the memory element becomes less dependent on how the user operates the device.

In some embodiments, the memory element, when assuming the fired position, controls a secondary function of the autoinjection device, wherein said secondary function is distinct from the function associated with the release of the plunger, i.e. the release being provided by the retaining element being moved in the radial direction to release the engagement with the retaining geometry of the plunger. A non-exhaustive list of secondary functions may include one or more of controlling initialisation or generation of a feedback signal, i.e. conditional to the release of the plunger, such as a visible, audible or tactile signal, or generation of an electronic signal to be recorded or stored in electronic circuitry, wherein the electronic signal is responsive to the release of the plunger. Still other secondary functions may include a latch function, such as for locking a needle shroud in a particular position, i.e. conditional to the release of the plunger.

In some embodiments the memory element is axially movable, and wherein said at least one surface is inclined relative to said radial direction and so oriented as to induce axial movement of the memory element from the firing position to the fired position upon radial movement of the retaining element.

In other embodiments the memory element is rotationally movable, and wherein said at least one surface is inclined relative to said radial direction and so oriented as to induce rotation of the memory element from the firing position to the fired position upon radial movement of the retaining element.

In some embodiments the retaining element includes a retaining surface that engages a cooperating surface of the retaining geometry to retain the plunger in the pre-firing position, and wherein one or both of the retaining surface and the cooperating surface include(s) a surface being inclined relative to said radial direction so that distal movement of the plunger, upon initial release of the retaining engagement, induces radial movement of the retaining element to disengage the retaining surface from the cooperating surface of the retaining geometry.

In some embodiments the trigger element assumes a pre-firing position when the trigger element assumes the pre-firing condition, and assumes a firing position when the trigger element assumes the firing condition, and wherein the trigger element cooperates with the retaining element to initiate release of the retaining engagement when the trigger element assumes the firing position.

In some embodiments the trigger element defines said memory element, and wherein the trigger element is movable from the pre-firing position to the firing position, and further to the fired position.

In some embodiments, the trigger element is movable axially from the pre-firing position to the fired position, such as movable proximally from the pre-firing position to the fired position. In some embodiments, the firing position is positioned at an intermediary position between the pre-firing position and the fired position.

In other embodiments, the trigger element is rotationally movable from the pre-firing position to the firing position. In some embodiments wherein the trigger element defines said memory element the trigger element is rotationally movable from the pre-firing position to the firing position, and further rotationally movable into the fired position. In these embodiments, the at one or both of the engagement surface and the activation surface include(s) a surface being inclined relative to said radial direction and being so oriented that radial movement of the retaining element induces rotation of the trigger element. The sequence of the rotation is initiated by the user which initially drives the trigger element to rotate from the pre-firing position to the firing position. Thereafter, the trigger element is in the first place urged to move by distal movement of the plunger, as forced by the energy source, which in turn induces radial movement of the retaining element, and which in turn induces rotation of the trigger element from the firing position to the fired position.

Some embodiments of the autoinjection device comprises a needle shroud being axially movable relative to the housing, and a needle shroud spring which is arranged biasing the needle shroud in the distal direction, wherein the needle shroud is movable from a first distal extended position into a proximal collapsed position when a proximally directed force is applied to the needle shroud, and from the proximal collapsed position into a distal extended locked position, and wherein the trigger element couples to the needle shroud so that the trigger element moves from the pre-firing position to the fired position in response to the needle shroud being moved from the first distal extended position into the proximal collapsed position.

In some embodiments, the first distal extended position is the same as the distal extended locked position. In other embodiments, the first distal extended position may be located distally or proximally relative to the distal extended locked position. The distal extended locked position defines a state wherein the needle shroud protects the needle from being touched by the user. The proximal collapsed position defines a state wherein the needle extends distally beyond the needle shroud, or where the needle is positionable to extend distally beyond the needle shroud, to allow for insertion of the needle into an injection site.

The needle shroud may be configured so that when it moves from the first distal extended position towards the proximal collapsed position the needle shroud causes the trigger element to move from the pre-firing position into the fired position, the needle shroud slaving the trigger element into the firing position, and optionally into the fired position.

In some embodiments a latch is associated with the trigger element, the latch engaging when the trigger element assumes the fired position to arrest the trigger element in the fired position.

The latch may in some embodiments be provided by cooperating latch geometries of the trigger element and the housing to prevent the trigger element from being moved distally away from the fired position. In other embodiments the latch is configured to rely on a frictional coupling, such as a frictional engagement, between the trigger element and the housing to prevent the trigger element from being moved distally away from the fired position. In some embodiments the latch is configured to provide a permanent axial locking of the trigger element relative to the housing when the trigger element assumes the fired position.

In some embodiments, when the needle shroud moves from the proximal collapsed position into the distal extended locked position, the needle shroud moves relative to the arrested trigger element, and wherein the needle shroud cooperates with the trigger element to lock the needle shroud as the needle shroud is moved distally into the distal extended locked position.

In some embodiments at least one of the needle shroud and the trigger element comprises a lock element which is resiliently urged towards the other of the needle shroud and the trigger element to move along relative to a surface of said other of the needle shroud and the trigger element when the needle shroud moves relative to the arrested trigger element until the lock element reaches a locking geometry formed in or on said other of the needle shroud and the trigger element upon the needle shroud being moved distally into the distal extended locked position so as to lock the needle shroud in the distal extended locked position.

In some embodiments the trigger element includes a distally directed lock surface configured to engage a proximally directed locking geometry of the needle shroud to prevent the needle shroud to be moved proximally when the needle shroud assumes the distal extended locked position.

In some embodiments the distally directed lock surface is formed on a resiliently movable lock element being movable from a non-locking position into a locking position, and wherein a biasing means urge the resiliently movable lock element towards moving to the locking position, and wherein the resiliently movable lock element is moved from the non-locking position into the locking position upon the needle shroud being moved from the proximal collapsed position into the distal extended locked position.

In some embodiments the resiliently movable lock element is configured to slide along a sliding surface of the needle shroud as the needle shroud is moved from the proximal collapsed position into the distal extended locked position for the distally directed lock surface of the trigger element to axially align with the proximally directed locking geometry of the needle shroud to enable the distally directed lock surface to engage with the proximally directed locking geometry.

In some embodiments the resiliently movable lock element is structured as a lock sleeve.

Some embodiments of the autoinjector forms a device wherein the energy source comprises a helical compression spring arranged in a pre-tensed state exerting a distally directed force on the plunger.

In further embodiments a latch is associated with the memory element, the latch engaging when the memory element assumes the fired position to arrest the memory element in the fired position.

The latch may in some embodiments be provided by cooperating latch geometries of the memory element and the housing to prevent the memory element from being moved distally away from the fired position. In other embodiments the latch is configured to rely on a frictional coupling, such as a frictional engagement, between the memory element and the housing to prevent the memory element from being moved distally away from the fired position. In some embodiments the latch is configured to provide a permanent axial locking of the memory element relative to the housing when the memory element assumes the fired position.

In some embodiments of the autoinjector, the device irreplaceably accommodates a container within the housing so that the container cannot be removed from the device without the use of tools. In such embodiments, the autoinjector forms a disposable device.

In some embodiments the container is provided as a syringe having a barrel and with an injection needle fixedly attached to the barrel.

In embodiments incorporating a cartridge and a separate needle unit, the cartridge and the needle unit may be initially held in a configuration where the cartridge and the needle unit are separated by a distance. The energy source may be capable, upon release of the plunger retaining arrangement, to cause the cartridge and the rear needle to enter into the state where the cartridge septum is pierced by the rear needle and subsequently to cause the plunger to move to dispense the drug through the needle.

In some embodiments the needle or needle unit substantially follows movement of the housing as the housing moves relative to the needle shroud. In particular embodiments, the needle or needle unit is attached to the housing in a way preventing relative axial movement between the housing and the needle.

As used herein, the term “drug” is meant to encompass any drug-containing flowable medicine or combinations of separately held plurality of drug-containing flowable medicines capable of being passed through a delivery means such as a cannula or hollow needle in a controlled manner, such as a liquid, solution, gel or fine suspension.

### DETAILED DESCRIPTION OF THE INVENTION

The invention will now be described in further detail with reference to the drawings in which:

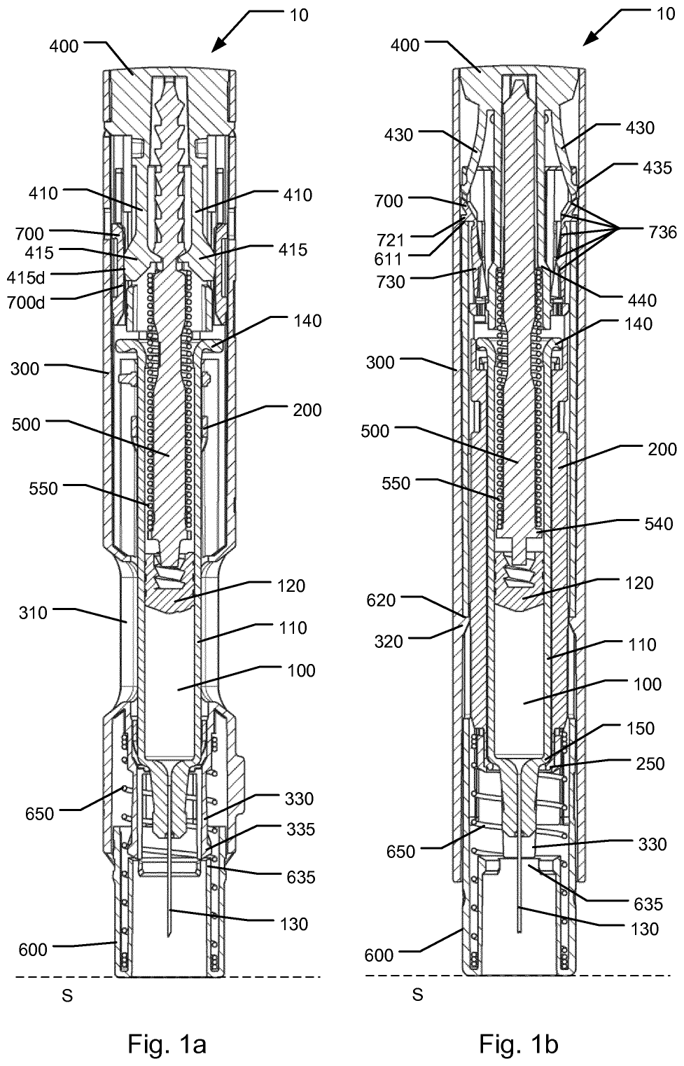

FIGS. 1a and 1b show sectional front and side views of an exemplary embodiment of an autoinjection device 10 according to the invention, the device being in a state where a needle shroud is fully extended and protects the needle of a held syringe,

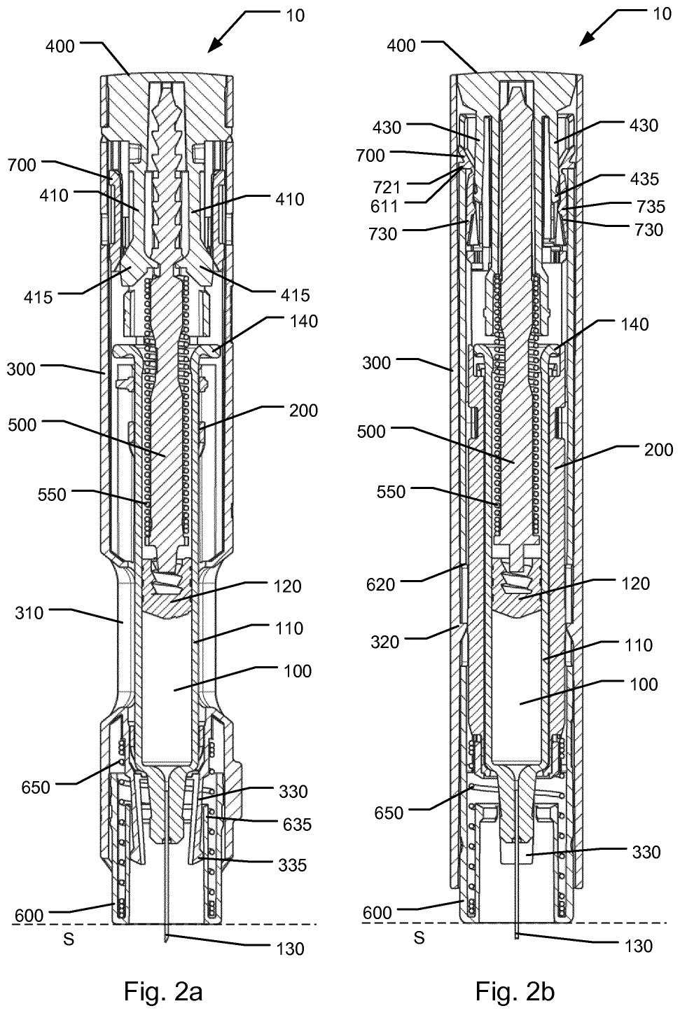

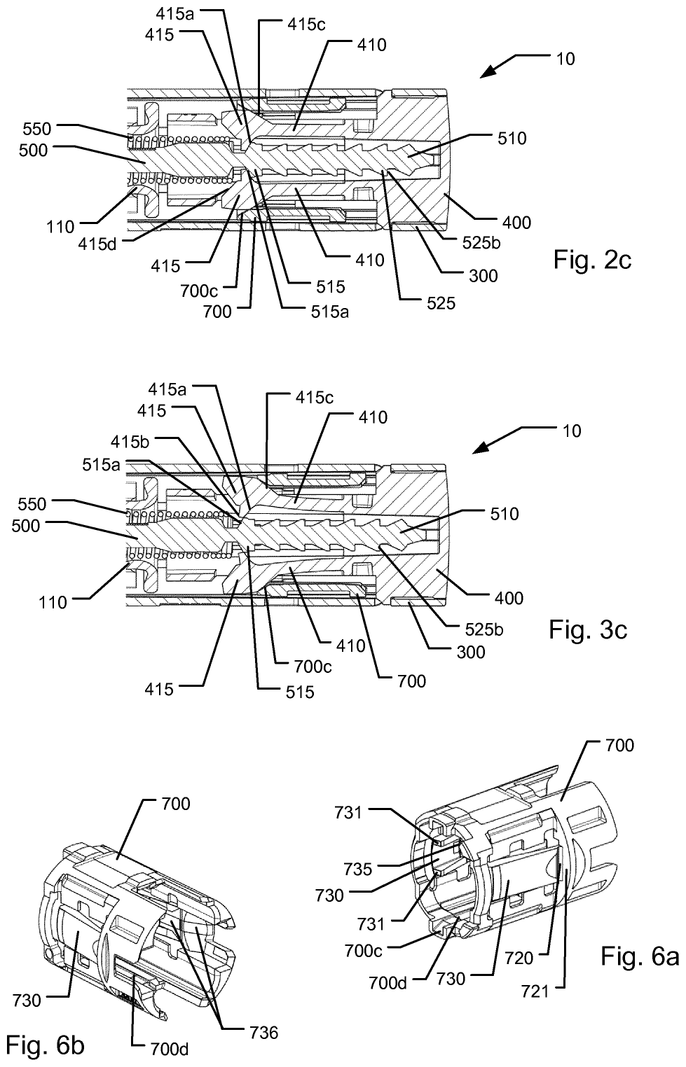

FIGS. 2a and 2b show sectional front and side views of the device 10 illustrating a state where the device has been pressed onto an injection site S and where an injection needle of a syringe initially protrudes from the needle shroud,

FIG. 2c is a detailed magnified view of FIG. 2a, showing the proximal portion of the device 10,

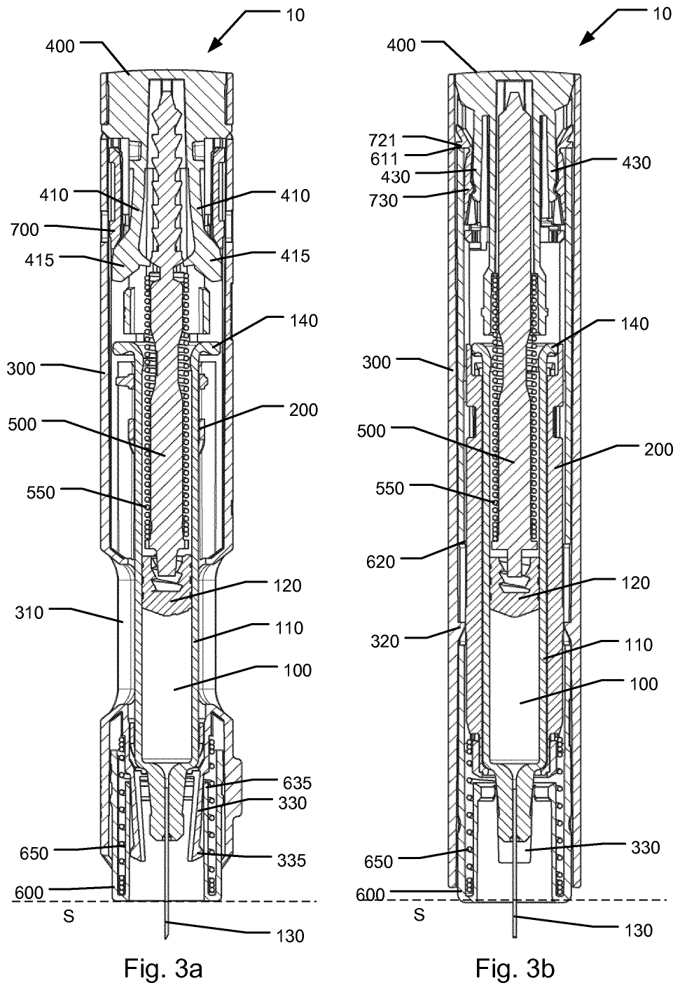

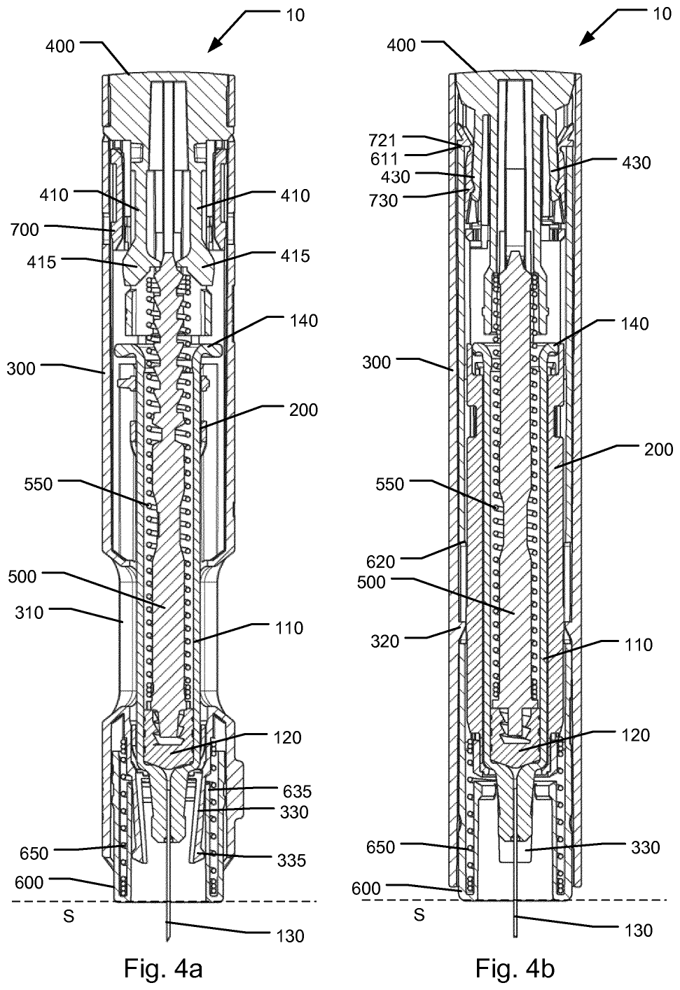

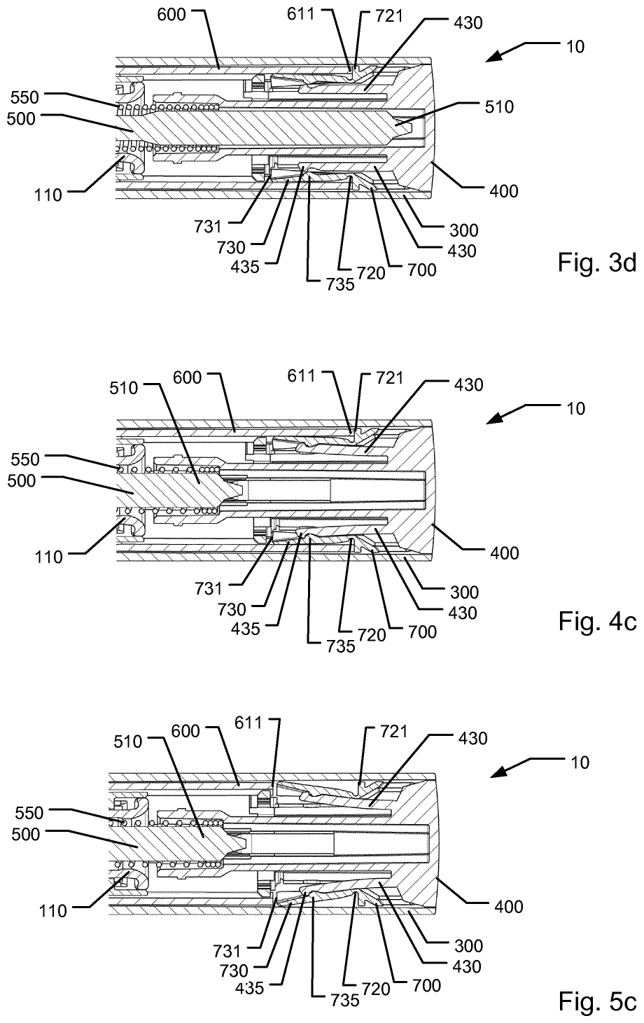

FIGS. 3a and 3b show sectional front and side views of the device 10 illustrating a state slightly before the needle protrudes fully from the needle shroud and wherein release of a plunger is about to be initiated,

FIG. 3c is a magnified view of FIG. 3a, showing the proximal portion of the device 10,

FIG. 3d is a detailed magnified view of the proximal portion of the device 10 in a view generally corresponding to the view shown in FIG. 3b but in a state just prior to the state shown in FIGS. 3a and 3b,

FIGS. 4a and 4b show sectional front and side views of the device 10 illustrating a state after the plunger has been released and where the drug of a held syringe has been expelled,

FIG. 4c is a detailed magnified view of FIG. 4b, showing the proximal portion of the device 10,

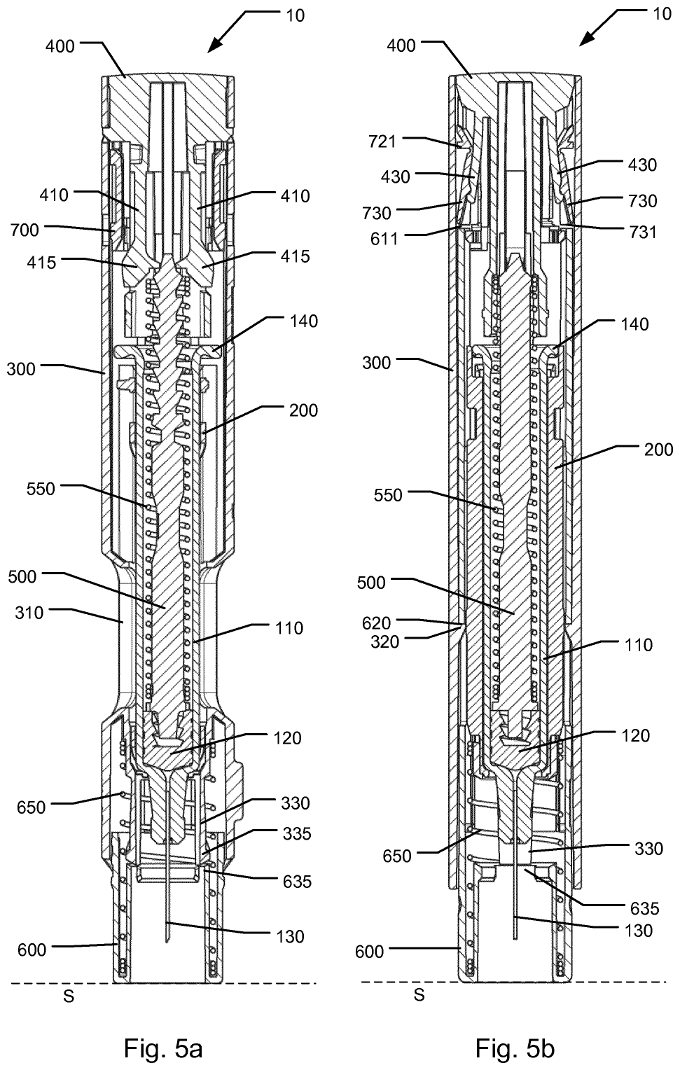

FIGS. 5a and 5b show sectional front and side views of the device 10 illustrating a state where the device has been lifted relative to the injection site S and wherein the needle shroud assumes a locked extended state,

FIG. 5c is a detailed magnified view of FIG. 5b, showing the proximal portion of the device 10,

FIGS. 6a and 6b show perspective proximal and distal views of trigger element 700 of the injection device 10, and

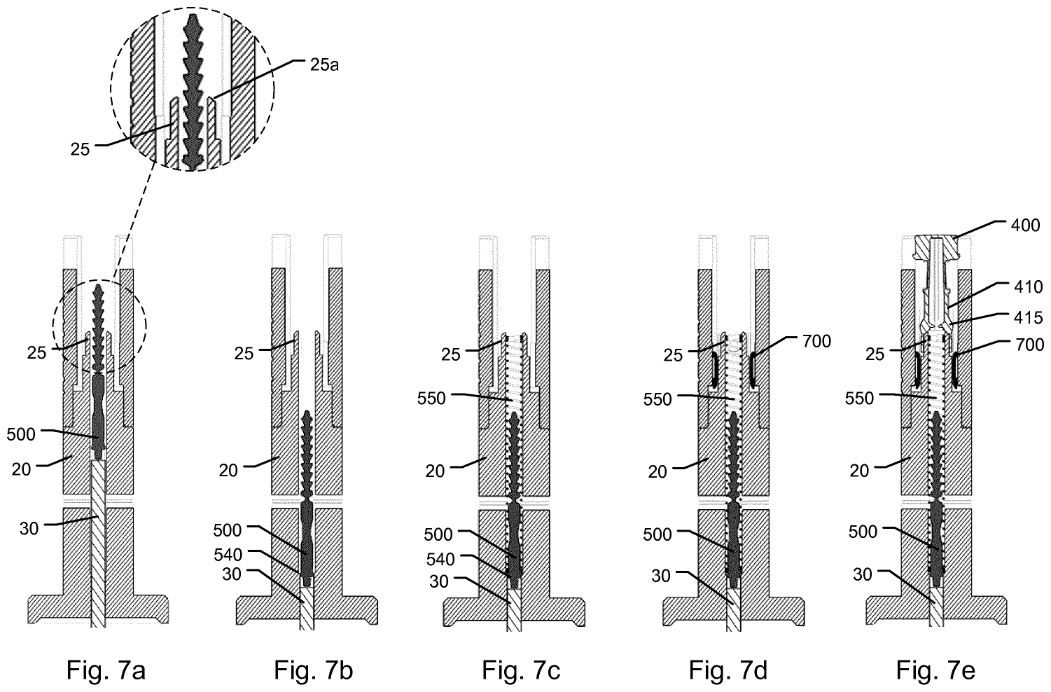

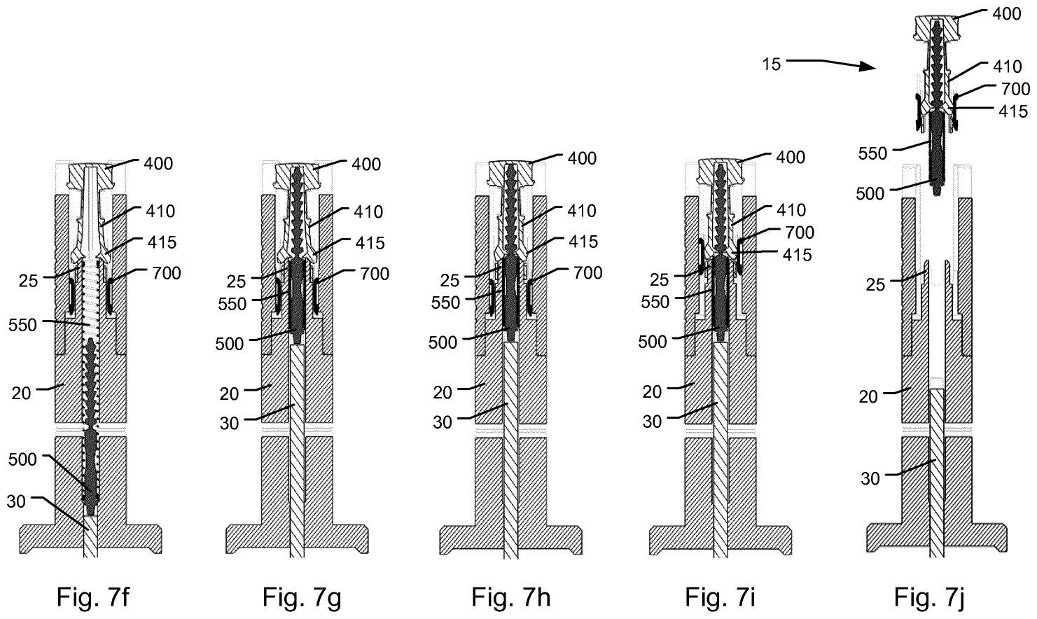

FIGS. 7a-7j are schematic views of an example method of preparing a power unit 15 of the autoinjection device 10, the individual views representing different states during assembly operations of the power unit.

### DESCRIPTION

In the context of the present disclosure it may be convenient to define that the term “distal end” in the appended figures is meant to refer to the end of the injection device which carries the injection needle whereas the term “proximal end” is meant to refer to the opposite end of the injection device pointing away from the injection needle. The shown figures are schematical representations for which reason the configuration of the different structures as well as the relative dimensions are intended to serve illustrative purposes only.

The following is a description of an exemplary embodiment of a medical injection device 10 for administering a pre-determined amount of a liquid medicament. The device 10 is a disposable autoinjector configured for expelling a dose of a drug in a single administration whereafter the device 10 is ready for disposal. FIGS. 1a through 5c show various states of the injection device 10 during operation thereof with different views offering a detailed assessment of the operating principle.

Referring to FIGS. 1a and 1b, injection device 10 includes an elongated housing 300 that extends along a central longitudinal axis, housing being configured for being gripped by the palm of the user. The housing 300 forms a tubular shell which is closed off at the proximal end by a cap which in the following will be referred to as a power base 400. During assembly the power base 400 snaps into the housing 300 by means of snap protrusions which are received in recesses or openings to provide a non-releasable mounting of power base 400 within the proximal end of the housing 300.

At the distal end of the housing 300 a protective cap (not shown) will normally be arranged to cover a needle arrangement located at the distal end of the housing.

In the shown embodiment, the housing 300 accommodates a standard prefilled syringe (PFS) as widely used in industry. The syringe 100 comprises a tubular barrel 110 having a neck portion 115 located distally wherein the neck portion 115 has a reduced diameter compared to the diameter of the barrel 100. An injection needle 130 is mounted to the neck portion 115 and a removable cap (not shown) provided in the form of a rigid needle shield (RNS) will prior to use be attached to the neck 115 so that the needle shield sealingly and sterilely seals off the needle 130 Internally in the barrel 110 a slideably arranged piston 120 is arranged. A drug may be accommodated within the barrel between the piston 120 and the needle 130. Although the shown syringe only incorporates a single piston 120, other configurations may incorporate multiple pistons for accommodation and expelling of one or more drugs, including drugs to be reconstituted before administration. In other not shown embodiments, instead of a PFS type syringe, the housing may alternatively include other types of medicament containers, such as cartridges configured to receive a separate injection needle.

Injection device 10 will typically be available in a form which further includes a removable protective cap (not shown) that attaches to a distal end of the device 10 to protect a needle end of the device 10. As commonly known for auto-injectors that incorporate a PFS syringe having an RNS shield attached, the protective cap may couple to the RNS so that the RNS is removed together with the protective cap. This situation is depicted in the state shown in FIGS. 1a and 1b.

In the shown embodiment, a syringe holder 200 is arranged to hold syringe 100 inside housing 300 in a manner so that syringe 100 is fixedly withheld within the housing 300 by means of the syringe holder 200. Syringe holder 200 includes a body extending along a central longitudinal axis and being adapted to receive the barrel 110 of syringe 100. The body of the syringe holder 200 includes two longitudinal body sections disposed around the central longitudinal axis, where each of the body sections has a distal end with a radial inwards flange section 250 adapted for being received in a circumferential gap between the shoulder section 150 of barrel and the not shown RNS covering the needle. In this way the syringe holder 200 retains the syringe 100 so as to prevent the syringe from moving distally relative to the syringe holder 200. The two longitudinal body sections of syringe holder 200 are connected to each other by means of flexible portions allowing the two body sections to be radially moved away from each other in order to insert the syringe with the RNS attached into syringe holder 200. During manufacture, the assembly formed by the syringe holder and the syringe with the RNS attached is insertable into housing 300 through a proximal opening in the housing shell.

The lower distal half of the housing 300 includes two opposing window openings 310 allowing visual inspection of the drug contained within the syringe of the device 10. In addition, window openings 310 allow a user of the device to determine whether or not the device 10 has been used for an injection by inspecting the presence or the location of a piston of syringe 100. In the course of an injection, window openings 310 also allow for a rod-shaped plunger 500 of the device to become increasingly visible by the plunger gradually blocking more and more of the space between window openings 310.

FIGS. 1a and 1b show front and side sectional views of the device 10 after the protective cap has been removed but in a condition prior to the administration operation. Shown protruding from the distal end of housing 300 is a needle shroud 600 which is received partly within and arranged coaxially and axially slidable relative to housing 300. a needle shroud spring 650 is arranged biasing the needle shroud 600 in the distal direction. Needle shroud 600 is movable, when a proximally directed force is applied to the needle shroud 600, from a first distal extended position (shown in FIGS. 1a and 1b) and into a proximal collapsed position (shown in FIGS. 4a and 4b). Upon release of the proximally directed force, the needle shroud spring 650 pushes needle shroud 600 from the proximal collapsed position into a distal extended locked position (shown in FIGS. 5a and 5b).

The injection device 10 is configured for being triggered to expel a dose when the needle shroud 600 is moved from the distal extended position towards the proximal collapsed position. As the syringe 100 is substantially fixedly mounted within housing 300 of the device 10, the injection needle 130 follows axial movement of the housing when the housing is moved relative to the needle shroud 600.

The protective cap, when attached to injection device 10, prevents the needle shroud 600 from being manipulated and thereby prevents premature unintentional triggering of the injection device 10. In the shown embodiment, this function may be provided by a mechanism incorporating radially flexible arms 330 formed in the housing, the flexible arms having heads 335 formed at an internal location in the housing 300 arranged to cooperate with an internal skirt 635 provided inside the needle shroud 600. The heads 335 and skirt 635 are located at the same radial position. Thus, for the needle shroud 600 to become pushed proximally relative to housing 300, the flexible arms 330 with heads 335 are required to become deflected radially inwards by cooperating with skirt 635 before the skirt and thus the entire needle shroud is movable away from the distal extended position. As long as the RNS and/or the protective cap is still attached to the syringe the flexible arms 330 with heads 335 are initially blocked against moving radially inwards by the presence of the RNS. The skirt 635 is thus not able to axially pass the heads 335. Only after removal of the protective cap with the RNS and forcing the needle shroud 600 towards the proximal collapsed position the heads will cooperate with skirt 635 to move the heads radially inwards and allow the skirt 635 to pass the heads of the flexible arms (cf. FIG. 2a).

Piston 120 is driveable towards the needle outlet in order to dispense medicament from the syringe 100. The dispensing is carried out by an expelling assembly incorporating the plunger 500 and a pre-stressed drive spring 550.

In the shown embodiment, the needle shroud 600 forms a distal portion and a proximal portion. The distal portion is provided as a generally hollow tubular member having a distal end rim arranged to form an abutment surface, the tubular member initially covering the injection needle 130. The proximal portion of the needle shroud 600 forms two opposed axial running legs extending from the distal portion and in the proximal direction for a substantive part of the length of the housing. Each of the two opposed axial running legs ends in a proximally facing abutment surface 611. The needle shroud 600 with its two legs is shaped to be accommodated within the housing 300 with the radial outer surface of the needle shroud being in intimate but slideable contact with a radially inwards facing cylindrical surface of the housing shell.

The needle shroud 600 cooperates with a trigger element 700 which is located at the proximal end of the needle shroud 600. Trigger element 700 serves as a memory element which assumes a first distal position prior to use of the device 10, and which assumes a second proximal pre-defined parked position after the device 10 has been fully triggered, and wherein the memory element stays in the parked position subsequent to triggering. In the shown embodiment the trigger element both serves as a trigger sleeve, and also serves as a lock sleeve for the needle shroud. For accommodating both functions, the trigger element 700 is movable axially in the proximal direction relative to the housing 300 from a pre-firing position (FIGS. 1a and 1b) to a firing position (FIGS. 2a and 2b), and further to a fired position (FIGS. 4a and 4b). Trigger element 700 is formed as a generally tubular hollow member. Initially, before triggering, and until the needle shroud 600 is pushed distally after expelling, a proximal portion of the legs of the needle shroud 600 is arranged in axially overlapping relationship with the trigger element 700. In this relative position the trigger element 700 is accommodated radially between the two opposed axial running legs of the needle shroud 600 so that an inner surface of each leg is in slideable contact with an internal surface of the trigger element 700. Referring to the state shown in FIG. 1b, and also referring to FIG. 6a, each proximally facing abutment surface 611 of the legs of the needle shroud 600 abuts a distal abutment surface 721 of the trigger element. Thus, when the needle shroud

In the shown embodiment, both the needle shroud 600 and the trigger element 700 are mounted in a way that prevents rotational movement but allows axial movement relative to the housing 300. The needle shroud 600 is urged in the distal direction by means of the needle shroud spring 650 so that when no externally applied force is exerted on the needle shroud, the needle shroud assumes its distal extended position which is shown in FIGS. 1a and 1b. In this position a stop geometry 620 on needle shroud 600 engages a stop 320 in the housing preventing the needle shroud 600 from moving further in the distal direction. When an externally applied force is exerted on the needle shroud 600 for moving the needle shroud in the proximal direction relative to the housing, such as when device 10 is pressed with the needle shroud against an injection site, the externally applied force acts counter to the force provided by the needle shroud spring 650 resulting in the needle shroud 600 and the trigger element 700 being forced to move in the proximal direction relative to the housing. When the needle shroud 600 assumes the proximal collapsed position a proximal facing surface of the trigger element 700 prevents the trigger element and thus the needle shroud 600 from moving further proximally relative to the housing.

As the device 10 is removed from the injection site, the needle shroud 600 will move distally due to the force from the needle shroud spring 650. After an injection has been performed, as the needle shroud 600 reaches its distal extended position again, as shown in FIGS. 5a and 5b, it will be locked in this position to render the needle shroud inoperable (to be further explained below). While referring to “its distal extended position” it is to be noted that the shown device 10 is so designed that the said distal extended position where the needle shroud is made inoperable corresponds to the initial distal extended position the needle shroud assumes prior to triggering. However, in other embodiments, the final distal extended position where the needle shroud is made inoperable may be located slightly different than the initial distal extended position prior to triggering, e.g. positioned at a position slightly proximally or slightly distally relative to the distal extended position shown in FIGS. 1a and 1b.

The needle 130 of syringe 100 is arranged at the distal end of the housing 300, such that the needle shroud 600 completely covers the needle when the needle shroud is in its distal extended position. When the needle shroud 600 is in its proximal collapsed position, the needle 130 protrudes through a central opening in the needle shroud 600.

The expelling assembly of injection device 10 is based on a plunger that is driven in the distal direction along the central longitudinal axis of the device for advancing the piston 120 to thereby expel the dose of drug accommodated within the syringe 100. The plunger 500 in the shown embodiment forms a solid rod having a circular flange arranged at the distal end of the plunger. In device 10 with the rod-shaped plunger 500 arranged along the central axis, a stored energy source in the form of a pre-stressed helical compression drive spring 550 is arranged to encircle the plunger rod 500 along a portion of its length. Drive spring 550 is energized by straining the compression spring during manufacture of the device. The distal end of drive spring 550 is supported onto plunger 500 by a circular flange arranged at the distal end of the plunger. The proximal end of drive spring 550 is supported by a spring seat (non-referenced) formed at a distal end of power base 400 and thus grounds the proximal end of drive spring relative to the housing 300.

As mentioned, in the shown embodiment, the drive spring 550 urges the plunger 500 in the distal direction. In the non-triggered state of the injection device 10, a plunger retaining arrangement associated with the housing engages with a retaining geometry of the plunger to retain the plunger 500 in a pre-firing position. In the shown embodiment, and referring to 1a, FIGS. 2c and 3c, the retaining arrangement comprises, on the plunger 500, a pair of stepped blocking geometries 515 proximally adjoining a recessed portion of the plunger 500.

The plunger retaining arrangement further comprises two retaining elements in the form of two retaining arms 410 extending axially in the distal direction from the proximal portion of the power base 400. Each of the two retaining arms 410 forms a radially resilient arm that ends in an enlarged blocking head 415 having its radially inwards facing portion situated in the recessed portion of the plunger rod 500. Generally referring to FIG. 2c, with the plunger retaining arrangement assuming the state shown in FIG. 1a, an inclined proximal surface 415a of enlarged blocking head 415 engages a correspondingly inclined distal surface 515a, these surfaces thus forming retaining geometries of plunger 500. Hence, the force exerted by drive spring 550 acts to push the enlarged blocking heads 415 radially outwards. In the initial non-triggered state of the device 10, as shown in FIG. 1a, a radially outward facing surface provided on each of the enlarged blocking heads 415 engages a radially inwards surface 700d of the trigger element 700, the presence of the trigger element, when located in the pre-firing position, thus effectively prevents triggering of device 10.

Referring to FIG. 2c, each of the enlarged blocking heads 415 includes, at a radially outwards portion thereof, an inclined proximally facing surface 415c configured for sliding engagement with a corresponding inclined distal facing surface 700c provided at the distal end of the trigger element (see FIGS. 2c and 6a). During triggering, the trigger element 700 will initially be pushed proximally by the needle shroud 600, due to engagement between elements 611 and 721, and once the trigger element assumes the firing position shown in FIG. 2c, each of the proximally facing surfaces 415c of enlarged blocking heads 415 axial aligns and engages the respective inclined distal facing surfaces 700c of trigger element 700. Due to the inclination, the radially outwards force generated by the bias of the drive spring onto the plunger is transferred to the enlarged blocking heads 415 which in turn will act to urge the trigger element 700 further proximally meaning that the force provided by needle shroud 600 to initiate the trigger movement of trigger element 700 will be amplified by the force provided by the drive spring 550. This causes the trigger element to be effectively pushed proximally until the trigger element 700 assumes the fired position shown in FIG. 3c. During movement of trigger element 700 from the firing position shown in FIG. 2c and into the fired position shown in FIG. 3c, the axial movement of trigger element is likely to be accompanied by proximal movement of the needle shroud 600 due to the momentum of movement of the needle shroud during the triggering movement of needle shroud.

As shown in FIG. 3c, the state refers to a situation where, for each resilient arm 410, the inclined proximal surface 415a of enlarged blocking head 415 slips free from engagement relative to the inclined distal surface 515a on the plunger 500, and the plunger 500 is thus released to be driven forward by the drive spring 550.

Alternatively to using a pre-stressed spring which is compressed during manufacture of the device, other embodiments of autoinjectors may include a mechanism for compressing the spring as an initial procedure when putting the device into use. Also, the energy source may in other embodiments be provided as a torsion spring which is pre-stressed to exert a torsion force for driving forward a rotational drive of the expelling assembly. Alternatively, the energy source may be in the form of a compressed medium such as a gas. Still alternatively, the energy source may include a gas generator such as an electro-chemical cell.

Referring again to FIG. 2c, the plunger 500 furthermore provides, at its proximal portion, a series of click protrusions formed as teeth 525, each tooth having a gradually rising slope and an abrupt decline 525b in the direction of relative movement. A radially inwards facing surface of the enlarged blocking head 415 of each resilient arm 410 is configured to sequentially cooperate by the inherent elastic properties of the resilient arm with each tooth 525 to generate an audible click as the enlarged blocking head passes each tooth. In the shown embodiment, each of the enlarged blocking heads 415 cooperate with six consecutive teeth. The enlarged blocking heads and the teeth thus generate progress clicks in the course of the dispensing procedure to signal expelling of liquid drug, and with the omission of a click to signify end of dosing. In the shown embodiment, two opposed retaining arms are provided in a symmetrical configuration, wherein the retaining arms cooperate with a corresponding number of protrusions or recesses formed on the plunger. In other embodiments a single arm may be provided necessitating a support surface of some kind arranged radially oppositely to the single arm. In still other embodiments, three or more arms may be provided, preferably being disposed symmetrically around the axis. In the shown embodiment, the plunger 500 is formed with surfaces configured for cooperation with engaging sliding surfaces of the power base 400, wherein the engaging surfaces are formed in a way that ensures that the plunger will not rotate relative to the power base at least as long as the click protrusions 525 of the plunger generate click sounds during expelling.

In the following, the components that relate to the needle shroud lock function will be further described. Referring back to FIG. 1b, 5c and FIG. 6a/6b, the trigger element 700 includes two resiliently movable lock elements formed as a pair of deflectable lock arms 730 forming part of the needle shroud lock mechanism. When the needle shroud lock function is established, the deflectable lock arms 730 render the needle shroud 600 permanently arrested, i.e. when the needle shroud, subsequent to finalization of an injection, is returned to the distal extended position. As shown in FIG. 6a, each of the deflectable arms 730 connects by means of a film hinge 720 to the remaining of the trigger element 700. Each of the deflectable arms 730 comprises a rigid beam section extending from the film hinge 720 to a free distal end comprising distally directed lock surfaces 731. The deflectable arms 730 are due to the film hinge 720 able to be moved radially outwards from a non-locking position where the locking arms 730 lie flush with the neighbouring surfaces of trigger element 700 and into a locking position where the lock surfaces 731 extend radially outwards from said neighbouring surfaces.

The needle shroud lock function further incorporates the power base 400. Power base 400 additionally includes two independent flexible arms 430 each extending in the distal direction from the power base. Each flexible arm is biased radially outwards so that a latch head 435 provided at the free distal end of the flexible arm assumes the position shown in FIG. 1b wherein the latch head 435 radially abuts the inner tubular surface of housing 300. Each latch head 435 comprises a radially outwards facing surface that is configured to cooperate by resiliently sliding against corresponding profiled axial tracks 736 of trigger element 700.

As shown in FIGS. 1b and 6a/6b, the deflectable arms 730 assume an unbiased non-locking radial position when the deflectable arms are not engaged by latch heads 435 of the two flexible arms 430. Each of the latch heads 435 of the two flexible arms 430 are configured to provide a radially outwards directed force on the corresponding deflectable arm 730 when the locking sleeve 700 is situated in the fired position, i.e. as shown in FIG. 4c. In this position each of the latch heads 435 grips behind a one-way latch protrusion 735 residing in the profiled axial track 736. Thus, when the trigger element 700 assumes the fired position the trigger element is arrested in the fired position and is prevented from moving distally again by latching engagement between latch heads 435 and one-way latch protrusions 735. For comparison the state shown in FIG. 3d provides a view of the device 10 just prior to the trigger element in the firing position where the latch heads 435 are positioned proximally relative to the one-way latch protrusions 735 and thus latch heads 435 are not yet latched. In both the firing position and in the fired position the flexible arms 430 of the power base 400 provides a radially outwards biased force onto the corresponding deflectable arm 730 of the trigger element. As the legs of the needle shroud 600 are positioned between the deflectable arms 730 and the housing 300 the deflectable arms 730 are still prevented from moving radially outwards into their locking position.

FIGS. 4a and 4b provides views of the device 10 in a state where the trigger element 700 is in the fired position, and the plunger 500 has already been caused to expel the dose of the syringe by plunging forward the piston 120 of syringe 100. The series of progression clicks have been generated during expelling and the piston has bottomed out in syringe barrel 100. The latch heads 435 of power base 400 grips behind the one-way latch protrusions 735 and. Thus, trigger element 700 is arrested in the fired position.

When the device 10 is removed from the injection site S the needle shroud spring 650 forces the needle shroud 600 from the proximal collapsed position into the distal extended locked position. During this movement, the proximally facing abutment surfaces 611 of the legs of the needle shroud initially moves out of engagement with the distal abutment surfaces 721 of the trigger element 700. Continued distal movement makes the legs of the needle shroud slide along the trigger element 700 until the proximally facing abutment surfaces 611 axially align with the distally directed lock surfaces 731 of the deflectable arms 730. Due to the radially outwards biased force from the flexible arms 430 onto the cooperating deflectable arms 730, the deflectable arms 730 are forced to move radially outwards into their locking position. As a consequence, the distally directed lock surfaces 731 of the deflectable arms 730 enter into blocking position relative to the proximally facing abutment surfaces 611 of the legs of the needle shroud 600 and the needle shroud 600 is prevented from moving towards the proximally collapsed position after the device 10 has been triggered.

Returning now briefly to details which relate to the triggering procedure of injection device 10 wherein the needle shroud 600 and the trigger element is moved in the proximal direction relative to the housing 300. Due to the profiled nature of axial tracks 736 the latch heads 435 initially climb a steep portion of the profiled axial tracks 736 (climb meaning move in the radial direction). This creates an initially high force which has to be overcome by the user when pushing device 10 against an injection site S to make the needle shroud 600 move towards the proximal collapsed position.

When the trigger element 700 is moved from the distal extended position towards the proximal collapsed position the two flexible arms 430 and the corresponding profiled axial tracks 736 of the trigger element 700 provide resistance to movement of the trigger element 700 and thus also resistance to movement of the needle shroud 600. Upon applying the autoinjector 10 at an injection site, a high axial reaction force is initially created when the flexible arms 430 engage the proximal end portion of the profiled axial tracks 736. Thus, a high force exerted on the needle shroud 600 is required in order for the flexible arms 430 to climb the profiled axial tracks 736. As soon as the flexible arms 430 have climbed the profiled axial tracks 736, resulting in the flexible arms 430 have been deformed radially inwards, the flexible arms 430 travel and slide along an almost constant height track profile as the needle shroud 600 is pushed further proximally relative to housing 300. This action requires considerable less force to be applied on the needle shroud 600 than the initial high force. Hence the needle shroud displacement will occur in two stages, i.e. a first high force stage and a second low force stage.

It will be appreciated, that the force needed for proximally displacing the needle shroud will be largely independent from the force provided by the drive spring but will rather be decided by the force of the needle shroud spring 650 and the force profile for the interaction between the flexible arms 430 and the profiled axial tracks 736. A further minor force which has to be overcome when pushing in the needle shroud 600 emanates from the flexible arms 330 of the housing cooperating with the inner tubular proximal rim 635 of the needle shroud 600, cf. the discussion mentioned above with respect to the removal of the protective cap/RNS.

As will be discussed further below, the above-mentioned firing position of trigger element 700, and the corresponding position of needle shroud 600, will be situated at the final part of the proximal needle shroud movement where the flexible arms 430 travel along the almost constant height profile of axial tracks 736. The high initial needle shroud force over a short distance assures that the needle shroud is fully displaced and the autoinjector is effectively triggered due to the inertia of the human motion.

Referring now to FIGS. 7a to 7j, these figures schematically depicts a series of cross-sectional side views of components for a power unit 15 and representative tools used for assembling the power unit. Each view represents a procedural step in a method of assembling the power unit. The power unit 15 represents an embodiment of a sub-assembly for inclusion into an autoinjector, such as the injection device 10 as discussed in relation to FIGS. 1a through 6b.

The power unit 15 is provided as an assembly of components which in the shown embodiment is made up by the following components: plunger 500, drive spring 550, power base 400 and trigger element 700. Once assembled to form a power unit the power unit 15 serves as a pre-assembled and pre-energized drive assembly to be inserted into housing 300 as part of assembling the pre-filled syringe 100 with the remaining components of injection device 10. The power unit 15, even though it holds the drive spring 550 in a compressed state, forms a stable assembly where the trigger element maintains the retaining elements of the power base securely engaged with the corresponding retaining surfaces of the plunger during storage and handling of the energized power unit.

Referring to FIG. 7a-7j, the power unit is assembled by means of a tool arrangement comprising multiple tool parts. A first tool part 30 is movably arranged along an axis to support and move the plunger when a plunger 500 is arranged coaxially with the axis. A second tool part 20 defines a jig that includes a central opening configured for slideably receiving the first tool part 30 inside the opening and for further receiving a held plunger supported by the first tool part 30. In the shown embodiment, the second tool part 20 is upwards open, i.e. it includes a proximally open area intended, in turn, to receive plunger 500, drive spring 550, trigger element 700 and power base 400. The second tool part 20 includes two arms 25 extending in the proximal direction configured to support a power base 400 when arranged onto the tool so that each of the axially extending retaining arms 410 formed on power base 400 rests on a respective arm 25 of the second tool part 20. The free end of each of the two arms 25 includes an inclined end section 25 configured to cooperate with a respective inclined distal facing surface 415d of each resilient arm 410.

The second tool part 20 further includes a generally cylindrical outer surface defined by the radially outer surface of the pair of arms 25 and an outer cylindrical tool portion (non-referenced). The latter parts are so shaped that a trigger element 700 can be held in a way that the radially outer surface of the pair of arms 25 is received within a held trigger element 700.

During assembly, in FIG. 7a, in a first step, a plunger 500 is firstly arranged within the central opening of the second tool part 20 so that the distal end of the plunger is supported by the first tool part 30 and so that a mid-section of the plunger is arranged between the two arms 25 of the second tool part 20. In a second step, the first tool part 30 is lowered thereby lowering the plunger 500 so that a free space is created between the two arms 25 (see FIG. 7b). Next, as a third step, the drive spring 550 is arranged to circumscribe the plunger 500 so that the distal end of the compression spring rests against the spring seat 540 of plunger 500 and so that the proximal end of drive spring extends almost to the free ends of the two arms 25 of the second tool part 20. The drive spring 550 is in this state axially uncompressed, as seen on FIG. 7c, and the distance between the two arms 25 is designed so that the drive spring fits between the two arms 25. Further, as a fourth step, a trigger element 700 is arranged so that the trigger element is positioned to encircle the two arms 25.

As shown in FIG. 7e, as a fifth step, the power base 400 is now arranged so that the two resilient arms 410 of power base rest onto the two arms 25 of the second tool part 20. By applying axial pressure between the power base 400 and the second tool part 20 the two resilient arms 410 are strained by being moved radially outwards into a tensed position. This radial movement is due to proximal facing inclined edge surfaces 25a formed on each of the free end of arms 25 and corresponding distal inclined surface portions 415d formed on the enlarged blocking heads 415 of each of the resilient arms 410.

In the shown embodiment, in a sixth step shown in FIG. 7f, the power base 400 is moved slightly in the distal direction, i.e. downwards, so that the resilient arms 410 are flexed sufficiently outwards for allowing the plunger 500 to be moved proximally, i.e. by allowing the abrupt declining surface 525b of each of the series of teeth 525 being able to pass the abrupt surface portions 415b formed on a distal facing surface of the enlarged blocking heads 415 of resilient arms 410. In accordance herewith, as a seventh step shown in FIG. 7g, the first tool part 30 is moved proximally towards the power base 400 meaning that the drive spring 550 will be axially compressed between the distal spring seat 540 on the plunger 500 and the proximal spring seat 440 formed in the power base (cf. FIG. 1b).

As shown in FIG. 7g, the plunger 500 is moved proximally relative to the power base 400 so that the enlarged blocking heads 415 of the resilient arms 410 axially aligns with the retaining geometries of plunger 500. As an eight step, shown in FIG. 7h, the power base 400 is moved slightly proximally allowing the natural resiliency of the resilient arms 410 to induce radial inwards movement of the arms 410 so that the inclined proximal surface 415a of each of the enlarged blocking heads 415 engages the correspondingly inclined distal surface 515a of plunger 500. In this position the drive spring 550 will be fully compressed and the first tool part 30 maintains pressure to keep this energy level of the drive spring.

As a ninth step, the trigger element 700 is moved proximally relative to the power base 400 into a position where the trigger element surrounds the enlarged blocking heads 415 of the resilient arms 41. In this position the radially outwards bias of resilient arms 410 emanating from the compressed drive spring 550 creates friction for retaining the trigger element in the pre-firing position referred to above in connection with FIGS. 1a and 1b. The two flexible arms 430 further act to releasably retain the trigger element 700 so that the trigger element will not accidentally move proximally from the pre-firing position. Although not shown, an additional mechanism may be provided, such as a detent mechanism or a one-way snap lock mechanism to allow the trigger element to be moved relative to the power base 400 from the position shown in FIG. 7h to the position shown in FIG. 7i but not vice-versa.

Finally, as shown in FIG. 7j, in a tenth step, the finished power module 15 may subsequently be removed from the tool arrangement facilitating storage of power module 15 and further assembly with additional components of the injection device 10.

It is to be noted that the above described embodiment where the principle of moving the resilient arms 410 outwards during assembly provides only one suitable mechanism for obtaining this process step. In alternative embodiments, as examples in accordance with the present invention, the distal facing surfaces 415d of enlarged blocking heads 415 may be provided with differently shaped surface geometries than the shape shown in FIGS. 2c, 3c and 7e though 7j. As an example for the straining surface geometry, the said distal facing surfaces of enlarged blocking heads 415 may be formed with stepped surfaces providing a radially inwards facing engagement surface configured for cooperating with a tool part that, during an assembly step, grips into the stepped surface and strains the enlarged blocking heads radially outwards from their resting position. The tool part may for example comprise first and second tool jaws that are movable radially outwards relative to each other to spread the enlarged blocking heads 415 apart. Further embodiments may include a tool part that may exhibit a non-circular outer surface, such as an oval shaped outer surface, at the area where the tool part engages with the enlarged blocking heads 415. Such tool may be introduced with a relatively narrow geometry being introduced between the enlarged blocking heads 415 and by subsequently rotating the tool part relative to the power base around the said axis engagement between less narrow portions of the tool and the enlarged blocking heads 415 may effectively urge the enlarged blocking heads radially outwards to allow the proximal movement of the plunger 500 relative to the power base 400. The tool is preferably arranged distally relative to a held power base. The tool part corresponding to the first tool part 30 described above will in certain embodiments include an opening which is shaped to allow the plunger with its protrusions to be moved through the opening such that the plunger is movable proximally relative to the held power base in order to bring the drive spring in the compressed state.

It is also to be noted that, although the shown embodiment shows an embodiment wherein the resilient arms 410 form click arms, thus serving both the function of retaining the plunger prior to firing as well as providing clicks during expelling, other embodiments may be provided wherein click arms are formed separately from retaining elements, such as separately from retaining arms. The instant invention may be utilized both for ensuring that click arms and/or retaining arms are tensioned radially outwards during an assembly step by using a suitable tool in a manner as disclosed above.

Some preferred embodiments have been shown in the foregoing, but it should be stressed that the invention is not limited to these but may be embodied in other ways within the subject matter defined in the following claims.

1. A power unit for use in an autoinjector,
the power unit configured for driving a plunger distally along an axis in an expelling movement so as to expel a drug from a held drug container, the power unit comprising:
a plunger having an elongated shape extending along said axis, the plunger comprising one or more retaining geometries and defining one or more radially protruding click protrusions,
a drive spring having a first end and a second end, the drive spring being arrangeable, in an assembly step for said power unit, in a tensed state wherein the first end of the drive spring acts on the plunger with a force biasing the plunger distally, and
a power base operably coupled to the drive spring and the plunger, the power base comprising a base part grounding the drive spring at a second end of the drive spring, and one or more retaining elements each releasably engaging a respective one of the one or more retaining geometries of the plunger to retain the plunger against the force of the drive spring the power base further comprising a resilient click arm configured to cooperate with said one or more click protrusions of the plunger, the click arm being moved radially to provide a click noise for each click protrusion that passes the click arm during the expelling movement,
wherein cooperating surfaces of the click arm and the respective click protrusions define, in the direction of the expelling movement, a leading surface pair that gradually increases tension in the click arm followed by a trailing surface pair that abruptly releases tension in the click arm to provide said click noise, and wherein the click arm further comprises a straining surface geometry arranged to cooperate, during said assembly step, with a tool arranged distally relative to the click arm for moving the click arm radially into a tensed state enabling the plunger to be moved proximally while allowing the surfaces of said trailing surface pair to pass each other during tensioning of the drive spring.

2. The power unit as defined in claim 1, wherein each of the surfaces of the trailing surface pair comprises an abrupt sloping surface, and wherein at least one of the surfaces of the leading surface pair comprises a gradually sloping surface.

3. The power unit as defined in claim 1, wherein the straining surface geometry of the click arm includes a distally oriented inclined surface having a surface normal inclined relative to the axis so that the surface normal intersects with the axis, and wherein said tool includes a reaction surface so oriented that, in the preparing step, the reaction surface engages the distally oriented inclined surface of the click arm thereby forcing the click arm to move radially outwards as the tool is moved proximally relative to the power base.

4. The power unit as defined in claim 1, wherein the power unit further comprises a trigger element being movable relative to the power base between a first position and a second position, wherein, when the trigger element assumes the first position, the trigger element engages with the one or more retaining elements to maintain retaining engagement between respective ones of the one or more retaining elements and the one or more retaining geometries of the plunger, and wherein, when the trigger element assumes the second position, the trigger element allows the one or more retaining elements to release said retaining engagement allowing the plunger to move distally.

5. The power unit as defined in any of the claim 1, wherein the plunger comprises a spring seat, and wherein the drive spring is a compression spring having the second end grounded by the power base and the first end providing a distally directed force on the spring seat of the plunger.

6. The power unit as defined in claim 1, wherein the power base comprises a transverse section and a spring guide that is arranged to extend axially in distal direction relative to the transverse section, the spring guide and/or the transverse section defining a distally facing spring seat that receives the second end of the drive spring, and wherein the click arm is formed in one piece with the transverse section and/or the spring guide.

7. The power unit as defined in claim 1, wherein the power base defines a distal facing spring seat that receives the second end of the drive spring, and wherein the power base defines a single unitary component having the spring seat formed in on piece with the click arm 414 and optionally, formed in one piece with the one or more retaining elements.

8. The power unit as defined in claim 1, wherein the power base is formed to define a closed proximal end cap configured for being coupled to the proximal end of a sleeve shaped housing of the autoinjector.

9. The power unit as defined in claim 1, wherein said click arm provides a first click arm and wherein the power base comprises one or more additional click arms configured similarly to the first click arm to cooperate with click protrusions of the plunger, and wherein the plurality of click arms are arranged symmetrically around the plunger.

10. The power unit as defined in claim 9, wherein respective ones of the one or more retaining elements is defined by one of said click arms.

11. The power unit as defined in claim 1, wherein the plunger is hollow, wherein the drive spring is a compression spring, and wherein the compression spring extends at least partly into the hollow plunger.

12. The power unit as defined in claim 1 wherein the drive spring is a helical compression spring encircling at least a portion of the plunger.

13. A method of assembling a power unit as defined in claim 1 for use in an autoinjector, wherein the method comprises the steps of:
a) providing the plunger, the power base, and the drive spring, wherein the drive spring is provided in the form of a helical compression spring,
b) inserting the plunger into a tool arrangement whereby the plunger is supported at its distal end by a first tool part,
c) arranging the drive spring concentrically with the plunger in axially overlapping relationship with the plunger,
d) arranging the power base concentrically with the drive spring with the straining surface geometry of the click arm being engaged by a second tool part,
e) moving the power base and the second tool part relative to each other to thereby move the click arm radially into a tensed state thereby enabling the plunger to be moved proximally while allowing the surfaces of said trailing surface pair to pass each other,
f) tensioning the drive spring between the power base and the plunger by moving the plunger in the proximal direction allowing the surfaces of said trailing surface pair to pass each other, and
g) arranging the one or more retaining elements in releasable engagement with the respective retaining geometries of the plunger to retain the plunger relative to the power base against the force of the drive spring.

14. The method of assembling a power unit according to claim 13, wherein the straining surface geometry of the click arm includes a distally oriented inclined surface having a surface normal inclined relative to the axis so that the surface normal intersects with the axis, and wherein said tool includes a reaction surface so oriented that, in the preparing step, the reaction surface engages the distally oriented inclined surface of the click arm thereby forcing the click arm to move radially outwards as the tool is moved proximally relative to the power base,
wherein in step d), the second tool part includes a reaction surface configured to engage said straining surface geometry of the click arm with the distally oriented inclined surface, and
wherein in step e), due to the engagement between the distally oriented inclined surface with the reaction surface, the click arm moves radially into a tensed state to enable the plunger to be moved proximally while allowing the surfaces of said trailing surface pair to pass each other.

15. The method of assembling a power unit according to claim 13, wherein the power unit further comprises a trigger element being movable relative to the power base between a first position and a second position, wherein, when the trigger element assumes the first position, the trigger element engages with the one or more retaining elements to maintain retaining engagement between respective ones of the one or more retaining elements and the one or more retaining geometries of the plunger, and wherein, when the trigger element assumes the second position, the trigger element allows the one or more retaining elements to release said retaining engagement allowing the plunger to move distally, and further comprises the steps of:
h) providing the trigger element and arranging the trigger element relative to the power base, and
i) subsequent to step g), moving the trigger element into the first position so that the trigger element engages with the one or more retaining elements to maintain retaining engagement between respective ones of the one or more retaining elements and the one or more retaining geometries of the plunger.
